(19) United States
(12) Patent Application Publication
(10) Pub. No.: US 2009/0204108 A1
(43) Pub. Date: Aug. 13, 2009

(54) DEVICE FOR INTRODUCING AND POSITIONING SURGICAL INSTRUMENTS AND CORRESPONDING METHOD

(76) Inventor: Rudolf Steffen, Bern (CH)

Correspondence Address: WRB-IP LLP, 1217 KING STREET, ALEXANDRIA, VA, 22314, US

(21) Appl. No.: 12/302,871

(22) PCT Filed: Jun. 21, 2006
(86) PCT No.: PCT/EP2006/063425

§ 371 Date: Nov. 28, 2008

(51) Int. Cl. A61B 17/00

(52) U.S. Cl. 606/1

(57) ABSTRACT

This invention relates to a device (20) for inserting and positioning surgical instruments (10) in the body of a patient, with an outer cover (22), into which outer cover (22) at least a front part (11) of the surgical instrument (10) is insertable, and which outer cover (22) is removable for positioning the surgical instrument (10) at the point of application, the outer cover (22) being pulled away from the front part (11) of the surgical instrument (10) by means of a pulling device (21). In particular, the outer cover (22) is able to be pulled backwards over the front part (21) <sic. (11)> of the surgical instrument (10), and the surgical instrument (10), or respectively its front part, is able to be uncovered again, so that the function assigned to the surgical instrument (10) is able to be carried out unimpeded. The outer cover (22) can be returned to the starting position, in particular also by means of a return device. The outer cover (22) is preferably able to be controlled by means of the pulling device (21) using the handle (15) of the surgical instrument (10).

### TECHNICAL FIELD

This invention relates to a device for inserting and positioning surgical instruments. In particular this invention relates to a device for insertion of surgical instruments into the body of a patient through a suitable body opening and their positioning at the point of application. In addition, this invention relates to a corresponding method.

### STATE OF THE ART

Surgical interventions in the human or animal body are a means of modern medicine used to facilitate a quicker recovery of the patient. These measures often make possible a complete healing of the disease or affliction, but also cause in themselves a relatively great trauma to the affected tissue, from which the body of the patient must subsequently recover. Many post-operative disorders can be attributed to the incisions in the skin and other soft tissues of the body. For this reason it has always been an aim of surgical treatment to cause as minimal discomfort as possible after the operation, i.e. to configure the surgical intervention in as gentle a way as possible.

Thus so-called laparoscopic surgery was introduced some time ago. With this surgical method, with the aid of an optical instrument, operative procedures are carried out through minimal incisions inside the abdominal cavity. This method was used initially for the surgical removal of the gall bladder, later also for carrying out more complex operations. The clear advantages of this surgical technique with respect to the recovery of the patient after the intervention led to the development of so-called minimally invasive surgical methods also in other areas, so that these minimally invasive operating techniques today have already replaced many conventional operating methods (with more extensive incision). Thus thoracoscopic or bone-stabilizing procedures are carried out in this way, and also with thyroid operations there is a tendency toward minimally invasive approaches, although they have not yet become widely accepted.

Smaller incisions and more minimal injury to the soft tissue at the point of access usually result in lesser post-operative pain and usually also to a speedier complete recovery of the patient. In addition, laparoscopic or thoracoscopic surgical methods offer the possibility of carrying out a more detailed examination of the abdominal or thoracic region for diagnostic purposes, which would normally not be possible with a conventional approach. Moreover a laparoscopic examination is sometimes carried out prior to a more complex operation in order to ascertain the condition and thus to be able to plan the further procedure better, for example.

Used with the minimally invasive surgical methods are various surgical instruments developed specifically for this purpose. Among such surgical instruments, the so-called clip suture devices or staplers play a big role. These clip suture devices are complex medical devices, in which different functions have been combined into one. Thus, by means of a clip suture device, portions of diseased or injured organs can be removed (resection), incisions in organs and tissue can be made (transection), or connections between blood vessels, nerves and hollow organs (anastomosis) can be established. The advantage of these devices is in particular a quicker and more efficient operation since different operational phases can be carried out using a single instrument.

However, the conventional clip suture devices in particular usually have the drawback that they are often of relatively large construction, owing to their complexity. In addition, the front part of the clip suture device with the actual functional insert, but also front parts of other surgical instruments often have a very irregular shape, so that insertion of these surgical instruments into the body of the patient as well as precise positioning at the point of application often proves to be very difficult. Above and beyond this, there is the risk, for instance during penetration of the clip suture device, that the surrounding tissue is injured by the functional insert, which could in some cases considerably prolong the convalescence of the patient.

### DISCLOSURE OF INVENTION

It is therefore the object of the present invention to propose a new device for inserting and positioning surgical instruments and a corresponding new method, which do not have the drawbacks of the state of the art. It is in particular the object of the present invention to provide a device and a corresponding method which make possible a precise, simple and quick insertion of surgical instruments, for example a dip suture device, and their subsequent precise positioning in the body of the patient.

These objects are achieved according to the present invention through the elements of the independent claims. Further advantageous embodiments follow moreover from the dependent claims and the specification.

In particular, the objects of the invention are achieved in that in a device for inserting and positioning surgical instruments in the body of a patient, having an outer cover, in which outer cover at least a front part of the surgical instrument is insertable, and which outer cover is removable at the point of application for positioning the surgical instruments, so that <sic> the outer cover is able to be pulled away from the front part of the surgical instrument by means of a pulling device. Such a device has the advantage in particular that at least the front part of the surgical instrument during its insertion in the body of the patient is protected by the outer cover. Since the front part of many surgical instruments, such as, for instance, that of a clip suture device, often has an irregular shape, its insertion into the body of the patient and the precise positioning at the point of application is sometimes extremely difficult. Moreover, through the penetration of the surgical instrument, damage to the surrounding tissue can occur, which can lead to complications and to a longer post-operative convalescence of the patient. The outer cover solves this problem in that it covers completely or partially the front part of the surgical instrument to be inserted or respectively to be positioned, and thus makes possible an easier insertion. Of course this outer cover must be removed, however, to enable use of the surgical instrument; thus it can be pulled away from the front part of the surgical instrument by means of a suitable pulling device. The device according to the invention for inserting and positioning surgical instruments thus makes possible an easier and gentler insertion as well as a quick and precise positioning of the surgical instrument in the body of the patient.

In an embodiment, the outer cover is able to be pulled backwards over the front part of the surgical instrument by means of a pulling device. This embodiment variant has in particular the advantage, that, on the one hand, the outer cover is not pulled forwards or sideways, but backwards. The front part of the surgical instrument can thereby be released in the simplest way from this outer cover. On the other hand, during the pulling away, the outer cover is able to follow the course of the surgical instrument, whereby further potential injury to the surrounding tissue is able to be prevented.

In another embodiment variant, the outer cover is returnable to the starting position by means of a return device. This embodiment variant has in particular the advantage that, for instance after completion of the surgical intervention, the front part of the surgical instrument can be covered again by means of the outer cover before the surgical instrument is taken out of the body of the patient. Not only can the surgical instrument be thereby led considerably more easily out of the body of the patient, but further injury to the surrounding tissue can also be prevented.

In a further embodiment variant, the outer cover is automatically returnable to the starting position. This embodiment variant has in particular the advantage that the return of the outer cover to the starting position can take place automatically and does not have to be carried out by the surgeon. Thus the surgical intervention can be carried out with the released surgical instrument, with the outer cover being then automatically pulled over the front part of the surgical instrument again. The surgical instrument can be subsequently removed from the body of the patient in ah especially easy way.

In another embodiment variant, the outer cover is attachable to a shaft of the surgical instrument by means of a locking device. This embodiment variant has in particular the advantage that the outer cover can be secured in a fixed position after insertion of the surgical instrument into the body of the patient and after its being pulled back at its place of application. The outer cover can thereby be held in a fixed position in particular during the surgical intervention, so that the operation is not interfered with in any way. Moreover the outer cover according to this embodiment variant of the present invention can be pulled out of the body of the patient together with the surgical instrument only after the surgical invention. It is also thereby possible to save time, whereby the chances for a quick recovery of the patient can be further increased.

In still another embodiment variant, the front part of the outer cover is closed, whereby an opening can be created at the tip of the outer cover by means of the pulling device and/or the perforations. This embodiment variant has in particular the advantage that the front part of the surgical instrument can be completely protected in an especially advantageous way, and can be released from the outer cover only after the insertion of the surgical instrument in the body of the patient, for example. Furthermore the movement of the outer cover of the device for inserting and positioning surgical instruments can be facilitated in an especially easy way.

In a further embodiment variant, the outer cover is made of a noble metal and/or plastic of different degrees of hardness. This embodiment variant has in particular the advantage that both noble metal and plastic are already optimally well known and tested materials for manufacture of surgical instruments. In addition, both of these materials have especially advantageous characteristics which become evident in particular during use of the device for inserting and positioning surgical instruments in the body of the patient, and also completely fulfill the authorization regulations for surgical instruments.

In another embodiment variant, the outer cover is rotationally symmetrical. This embodiment variant has in particular the advantage that the position relative to the surgical instrument itself of the device according to this embodiment variant of the present invention for inserting and positioning surgical instruments in the body of the patient plays no role. Moreover, during insertion, the direction of penetration and/or the angle of penetration in the body of the patient can be changed without the insertion being thereby impeded or even prevented. Above and beyond this, a rotationally symmetrical shape of the outer cover can be manufactured in an especially advantageous way, whereby the overall costs can be reduced for the device for inserting and positioning surgical instruments in the body of the patient according to this embodiment variant of the present invention.

In still another embodiment variant, the outer cover has substantially an olive-shaped and/or conical form. This embodiment variant has in particular the advantage that this form is suitable in an especially advantageous way for insertion into the body of a patient. The surgical instrument can be guided to the point of application without great resistance. These special shapes make possible moreover an insertion with as little damage as possible to the surrounding tissue, which tissue is pushed aside during the penetration, owing to the increasing circumference from the tip of the outer cover, according to this embodiment variant.

In a further embodiment variant, using the handle and/or the operating handle of the surgical instrument, the outer cover is controllable by means of the pulling device. This embodiment variant has in particular the advantage that operations using the device for inserting and positioning surgical instruments in the body of the patient according to this embodiment variant of the present invention can be carried out in a conventional way. The practicing physicians do not have to be retrained to handle the device according to the invention, and moreover do not need to change their accustomed operating practices. Furthermore, the outer cover according to this embodiment variant can be monitored and controlled also during the operation, if necessary.

It should be stated here that, besides the device according to the invention, this invention also relates to a corresponding method for inserting and positioning surgical instruments in the body of a patient.

### BRIEF DESCRIPTION OF DRAWINGS

The embodiment variants of the present invention will be described in the following with reference to examples. The examples of the embodiments are illustrated by the following attached figures:

FIG. 1 shows a diagrammatical representation in perspective of a surgical instrument from the state of the art.

FIG. 2 shows a diagrammatical representation in perspective of a device for inserting and positioning surgical instruments in the body of a patient according to an embodiment variant of the present invention during insertion of the surgical instrument into the body of the patient.

FIGS. 3A and 3B show diagrammatically a cross section of two devices for inserting and positioning surgical instruments in the body of a patient according to two embodiment variants of the present invention.

FIG. 4 shows a diagrammatical representation in perspective of a device for inserting and positioning surgical instruments in the body of a patient according to an embodiment of the present invention during the surgical intervention.

### MODES FOR CARRYING OUT THE INVENTION

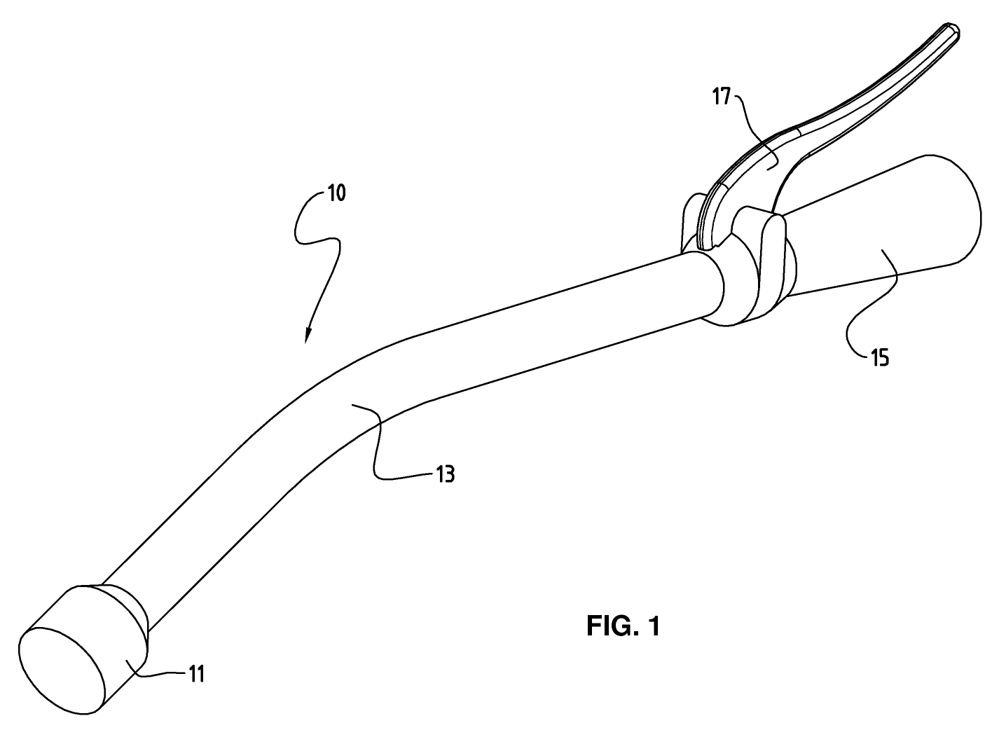

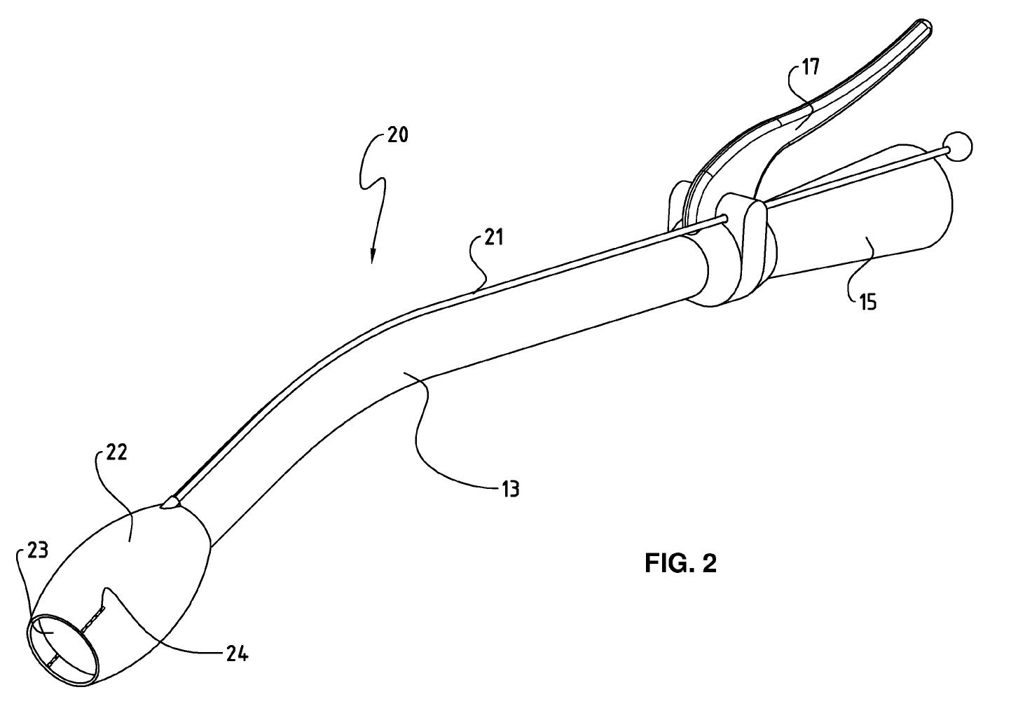

FIG. 1 shows a surgical instrument, to be more precise, a clip suture device from the state of the art. In FIG. 1, the reference numeral 10 refers to the surgical instrument itself, the reference numeral 11 to the front part of the surgical instrument 10, and the reference numeral 13 to the shaft of the surgical instrument 10. The front part 11 of the surgical instrument 10 can comprise in particular various functional inserts, which are able to execute various functions, and which can also have therefore different shapes and/or sizes. These functional inserts ban likewise be made of the most diverse materials, which can be the same or different from the materials of the surgical instrument 10 itself. These functional inserts can also in particular be exchanged, according to need, so that the surgical instrument 10 can be used for different tasks. Furthermore the reference numeral 15 in FIG. 1 refers to the handle of the surgical instrument 10, and the reference numeral 17 to the operating handle of the surgical instrument 10, which can serve the practicing physician to hold and control the surgical instrument 10, or respectively the corresponding functional insert on the front part 11 of the surgical instrument 10, during the surgical intervention. Illustrated in FIG. 2 is a device 20 for inserting and positioning surgical instruments 10 in the body of a patient according to an embodiment variant of the present invention during insertion of the surgical instrument 10 into the body of the patient. In FIG. 2, the reference numerals 13, 15 and 17 refer to the shaft, the handle and the operating handle of the surgical instrument 10, as in FIG. 1 in the preceding. Above and beyond this, the reference numeral 21 refers to a pulling device, and the reference numeral 22 to an outer cover. The outer cover 22 encloses the internal space 23, in which at least the front part 11 of the surgical instrument 10 with the functional insert belonging thereto can be accommodated. Finally, the reference numeral 24 refers to the perforations at the tip of the outer cover 22 of the surgical instrument 10. The pulling device 21 is connected to the outer cover 22, so that the outer cover 22 can be pulled backwards by means of the pulling device 21. The pulling device 21 can be designed, for example, as a thin strand made of the same material as the outer cover 22. Of course the pulling device 21 can also have a different form, however, or be made of a different material. In particular, the pulling device 21 can be connected to the handle 15 and/or to the operating handle of the surgical instrument 10, so that the outer cover 22 can be controlled from the handle 15 of the surgical instrument 10.

The outer cover 22 can have an olive-shaped form, for example, but can also be designed having a conical or other shape. In particular, the outer cover 22 can also be rotationally symmetrical, for example about its longitudinal axis. This special form for the outer cover 22 makes possible an especially easy insertion of the surgical instrument 10 into the body of a patient, as well as a simpler and therefore more economical manufacture. Nevertheless we emphasize here that both the outer cover 22 as well as the pulling device 21, or any other components of the device according to the invention for inserting and positioning surgical instruments 10 in the body of a patient, can of course have completely different shapes or modes of operation. Thus, for example, in particular the outer cover 22 on the front end can also be completely closed during the insertion of the surgical instrument 10. Also the pulling device 21 can be connected to the outer cover not only at a single place, but at a plurality of places at the same time.

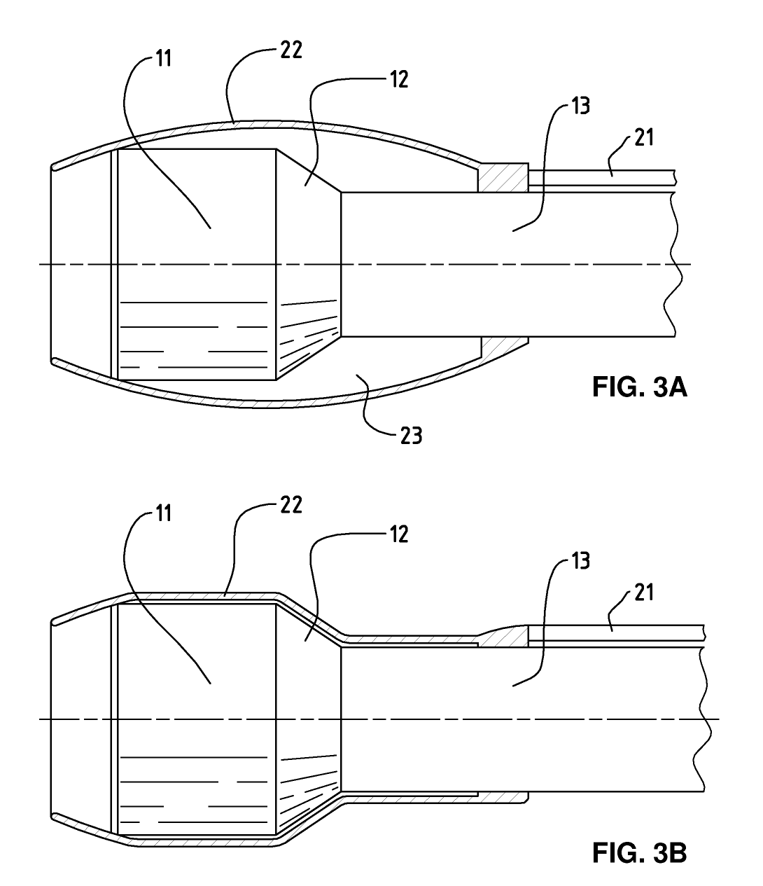

FIGS. 3A and 3B show two special embodiment variants of the front part of the device 20 for inserting and positioning surgical instruments 10 in the body of a patient. Also in these figures, the reference numeral 11 refers to the front part of the surgical instrument 10, the reference numeral 12 to the rear side of the front part of the surgical instrument 10, and the reference numeral 13 to the shaft of the surgical instrument 10. Furthermore the reference numeral 21 refers to the pulling device, which is connected to the outer cover 22 of the device for inserting and positioning surgical instruments 10. In FIG. 3A, the reference numeral 23 refers to the internal space that is formed by the outer cover 22. The outer cover 22 can in particular enclose the front part 11 of the surgical instrument 10 and a portion of its shaft 13 in each case. The outer cover 22 can thereby have in particular a shape in which its cross section at the tip is smaller than the cross section at a place that is closer to the shaft 13 of the surgical instrument 10. During penetration into the body of the patient to the point of application, it is thereby possible for the outer cover 22 to meet as little resistance as possible. As shown in FIG. 3A, the outer cover 22 can be bigger than the front part 11 of the surgical instrument 10 with the functional insert belonging thereto, so that an interim space is created between the outer cover 22 and the front part 11 of the surgical instrument 10. On the other hand, as in FIG. 3B, the outer cover 22 can be adapted to the shape of the front part 11 of the surgical instrument 10 in the most precise way. The outer cover 22 can have an opening at the tip, as shown, but can also be completely closed.

During insertion of the surgical instrument 10 into the body of the patient, the outer cover 22 serves as a kind of shield which, on the one hand, protects the sensitive front part 11 of the surgical instrument 10 with the functional insert belonging thereto against damage, and, on the other hand, supports in a positive way the insertion of the surgical instrument 10 into the body of the patient, thanks to its special form. Moreover the special form of the outer cover 22 also protects the surrounding tissue against injuries from the surgical instrument 10. After the surgical instrument 10 with the outer cover 22 has been guided to the point of application in the body of the patient, the pulling device 21 can be actuated, so that the traction is transmitted to the outer cover 22 connected to the pulling device 21. The outer cover 22 can have one or more perforations 24 at the tip, for example, which are cracked open by the traction effect of the pulling device 21, and make possible the movements of the outer cover 22. Of course other embodiment variants are also possible and conceivable. Thus, by means of the pulling device 21, the front part 11 of the surgical instrument 10 can be released from the outer cover 22. Then the surgical instrument 10 can be used in the accustomed way. After being pulled away from the front part 11 of the surgical instrument 10, the outer cover 22 can be removed from the body of the patient on the same path, for example, or can be placed in the vicinity of the surgical instrument 10 during the surgical intervention and can be taken out of the body of the patient only later, together with surgical instrument 10 itself.

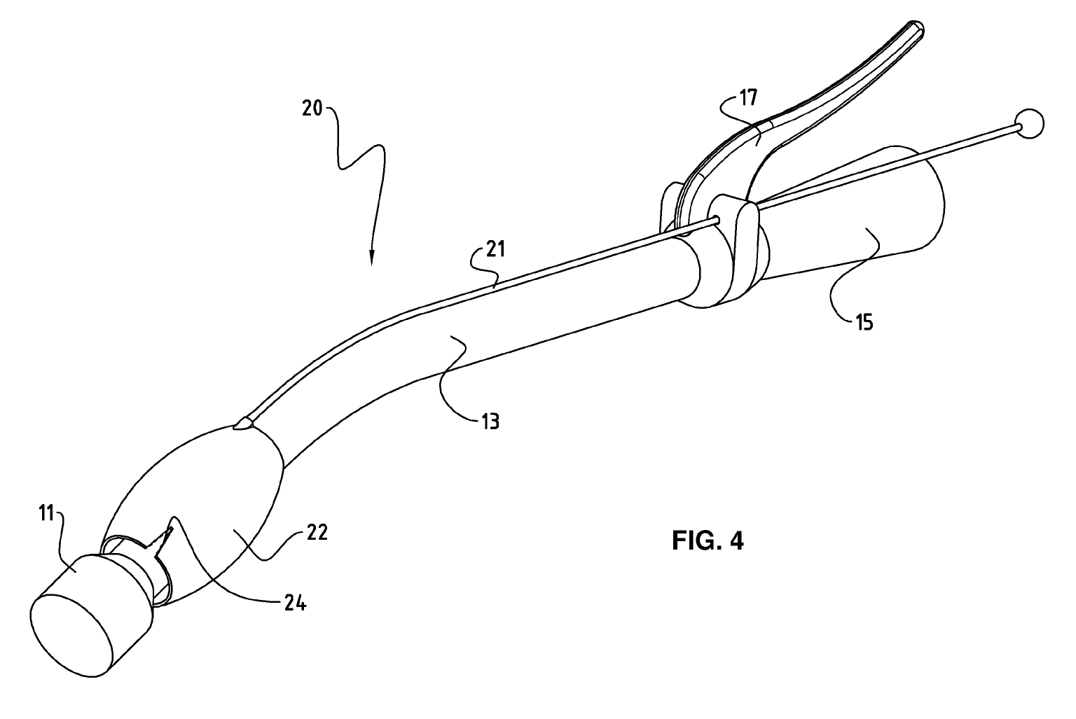

Preferably, however, after release of the front part 11 of the surgical instrument 10, the outer cover 22 can be pulled backwards, i.e. over the shaft 13 of the surgical instrument 10. A device 20 for inserting and positioning surgical instruments 10 in the body of a patient according to this embodiment variant of the present invention is shown in FIG. 4. In FIG. 4, the reference numeral 11 refers again to the front part of the surgical instrument 10, the reference numeral 13 to the shaft of the surgical instrument 10, the reference numeral 15 to the handle of the surgical instrument 10, and the reference numeral 13 to the operating handle of the surgical instrument 10. The reference numeral 21 refers again to the pulling device, which is connected to the outer cover 22 of the device for inserting and positioning surgical instruments 10. The reference numeral 24, finally, refers to the perforation 24 at the tip of the outer cover 22.

The device 20 for inserting and positioning surgical instruments 10 in the body of a patient can also comprise in particular further components which are not shown in the attached drawings. Thus, for example, the outer cover 22 can be led back into the starting position by means of a return device. This return device can be designed as a strand made of suitable material, similar to the pulling device 21, for example, but can also be designed as a separate, more complex device. The device 20 for inserting and positioning surgical instruments 10 in the body of a patient can also comprise, for example, a device that can be used for automatic return of the outer cover 22 to the starting position. This return device can thereby be designed in particular as a mechanical spring, or as any other suitable device. Finally, the outer cover 22 can be fixed by means of a locking device, for example on the shaft 13 of the surgical instrument 10, so that it cannot slide during the surgical invention and cause damage.

In conclusion, it is to be pointed out that the embodiment variants described here by way of example represent only a selection of possible ways of carrying out the inventive concept, and should in no way be seen as limiting. One skilled in the art will understand that many other modes of implementation of the invention are possible without losing sight of the essential features of the invention.

1-17. (canceled)

18. A device for inserting and positioning surgical instruments in the body of a patient, with an outer cover, into which outer cover at least a front part of the surgical instrument is insertable, and which outer cover is removable at the point of application for positioning the surgical instrument, wherein
an opening is able to be created at the tip of the outer cover by means of a pulling device and/or perforations, and
the outer cover is able to be pulled backwards, in one piece, over the front part of the surgical instrument by means of the pulling device.

19. The device according to claim 18, wherein the outer cover is able to be returned to the starting position by means of a return device.

20. The device according to claim 19, wherein the outer cover is able to be returned automatically to the starting position.

21. The device according to claim 18, wherein the outer cover is attachable to a shaft of the surgical instrument by means of a locking device.

22. The device according to claim 18, wherein the front part of the outer cover is closed.

23. The device according to claim 18, wherein the outer cover is made of a noble metal and/or plastic of different degrees of hardness.

24. The device according to claim 18, wherein the outer cover is rotationally symmetrical.

25. The device according to claim 18, wherein the outer cover has substantially an olive-shaped and/or conical form.

26. The device according to claim 18, wherein the outer cover is controllable by means of the pulling device, using the handle and/or the operating handle of the surgical instrument.

27. A method for inserting and positioning surgical instruments in the body of a patient, at least the front part of the surgical instrument being inserted into an outer cover, the surgical instrument being inserted with the outer cover into the body of the patient, and the outer cover being removed at the point of application for positioning the surgical instrument, wherein
an opening is created at the tip of the outer cover by means of a pulling device and/or perforations, and
the outer cover is pulled backwards, in one piece, over the front part of the surgical instrument by means of the pulling device.

28. The method according to claim 27, wherein the outer cover is returned to the starting position by means of a return device.

29. The method according to claim 28, wherein the outer cover is automatically returned to the starting position.

30. The method according to claim 27, wherein the outer cover is attached to a shaft of the surgical instrument by means of a locking device.

31. The method according to claim 27, wherein the front part of the outer cover is closed

32. The method according to claim 27, wherein the outer cover is controlled by means of the pulling device, using the handle and/or the operating handle of the surgical instrument.
